(19) United States
(12) Patent Application Publication
(10) Pub. No.: US 2024/0424212 A1
(43) Pub. Date: Dec. 26, 2024

(54) AUTOMATICALLY OR MANUALLY INITIATED MEAL BOLUS DELIVERY WITH SUBSEQUENT AUTOMATIC SAFETY CONSTRAINT RELAXATION

(71) Applicant: INSULET CORPORATION, Acton, MA (US)

(72) Inventors: Rangarajan NARAYANASWAMI, Weston, MA (US); Yibin ZHENG, Hartland, WI (US); Mert SEVIL, Manchester, NH (US); William WHITELEY, Knoxville, TN (US); Saeed SALAVATI, Sugar Land, TX (US); Sam CARL, Waltham, MA (US)

(21) Appl. No.: 18/824,363

(22) Filed: Sep. 4, 2024

Related U.S. Application Data
(63) Continuation of application No. 18/390,338, filed on Dec. 20, 2023, now Pat. No. 12,097,355.
(60) Provisional application No. 63/478,842, filed on Jan. 6, 2023.

(51) Int. Cl. A61M 5/172

(52) U.S. Cl. CPC A61M 5/1723; A61M 2005/1726; A61M 2205/505; A61M 2230/201

(57) ABSTRACT

The exemplary embodiments may provide a drug delivery device that receives glucose level values for a user (e.g., a diabetic patient) and based on the glucose level values, determines when the user has consumed a meal. In some embodiments, the drug delivery device may calculate an appropriate bolus dose and automatically deliver the drug bolus to the user. In some embodiments, instead of detecting the meal, the user may announce the meal, such as by activating an element on the drug delivery device or on a management device for the drug delivery device. Responsive to the meal announcement, the drug delivery device may calculate the drug bolus dose and deliver the drug bolus. In conjunction with the delivery of the drug bolus, the drug delivery device may relax one or more safety constraints for a relaxation period following the drug bolus delivery so that additional basal drug may be delivered, if needed, under relaxed constraints.

### RELATED APPLICATIONS

This application is a continuation of U.S. patent application Ser. No. 18/390,338, filed Dec. 20, 2023, which claims the benefit of U.S. Provisional Patent Application No. 63/478,842, filed Jan. 6, 2023, the entire contents of which are incorporated herein by reference in their entirety.

### BACKGROUND

Diabetic patients need additional insulin to offset the rise in glucose levels that follows consumption of a meal. Traditionally, such diabetic patients have delivered insulin boluses via manual injections in anticipation of consuming meals. The diabetic patients must determine the doses for the insulin boluses. This can be difficult and prone to error. The diabetic patients must correctly determine the amount of carbohydrates in the meals and must correctly determine the proper insulin bolus doses to offset the rise in glucose levels that will result from consuming the calculated amounts of carbohydrates for the meals. The diabetic patients also must time the delivery of the insulin bolus properly relative to consuming the meals. Sometimes diabetic patients may even forget to deliver an insulin bolus for a meal. The net result often is poor glucose level management. Diabetic patients may become hyperglycemic as a result of choosing insulin boluses that are too small or may become hypoglycemic by choosing insulin boluses that are too large.

### SUMMARY

In accordance with a first inventive facet, a drug delivery system for delivery of a drug, such as insulin, to a user may include a storage or reservoir for the drug and a needle and/or cannula for piercing the skin of the user to deliver the drug from the storage. The drug delivery system may further include a fluid path for the drug between the storage to the needle or cannula and a non-transitory computer-readable storage medium storing computer programming instructions and other historical information. The drug delivery system may additionally include a processor configured to execute the computer programming instructions. Executing the computer programming instructions may cause the processor to constrain delivery of the drug to the, receive glucose level values for the user, and/or other analyte level values, and determine whether the user has eaten based on the received analyte level values for the user. Executing the computer programming instruction also may cause the processor to provide an auto bolus capability to deliver a first portion of a drug bolus to the user responsive to the processor determining that the user has eaten, and relax at least one of the drug safety constraints for a period following the delivery of the first portion of the drug bolus so that larger doses of basal drug deliveries may be delivered if needed without being subject to the at least one drug safety constraints that were relaxed.

The executing of the computer programming instructions may further cause the processor to deliver a second portion of the drug bolus. Executing the computer programming instructions may further cause the processor to determine a dose for the second portion of the drug bolus. The second portion of the dose may be determined based at least in part on a most recent received analyte level value and drug on board for the user. Executing the computer programming instructions may further cause the processor to prevent delivery of another drug bolus during a cool down period following delivery of the second portion of the drug bolus. The executing of the computer programming instructions may further cause the processor to cancel the auto bolus capability if an activity mode is set wherein the activity mode being set indicates that the user is active or going to be active shortly or if the user is in the cool down period. The executing of the computer programming instructions may further cause the processor to cancel the relaxing of the at least one drug delivery constraint if at least one cancelation condition other than the expiration of the period is satisfied. The cancelation condition(s) may include at least one of a difference between consecutively received blood glucose values for the user that exceeds a threshold or an activity mode is set wherein the activity mode being set indicates that the user is active or going to be active shortly.

In accordance with another inventive facet, a method configured to be executed on or performed by a processor of a drug delivery system may comprise receiving glucose level values for the user, and/or other analyte level values, and determining whether the user has eaten based on the received analyte level values for the user. The method may further comprise an auto bolus capability to determine a first portion of a drug bolus to be delivered to the user responsive to the method determining that the user has eaten, determining that the drug delivery system has delivered the first portion to the user, and relaxing at least one drug safety constraint for a period following the determination that the first portion of the drug bolus has been delivered, wherein relaxing the at least one drug safety constraint allows determination of larger doses of basal drug deliveries to be delivered if needed without being subject to the at least one drug safety constraints that were relaxed.

The method may further comprise determining a dose for the second portion of the drug bolus to be delivered to the user. The second portion of the dose may be determined based at least in part on a most recent received analyte level value and drug on board for the user. The method may further comprise determining that the second portion of the drug bolus has been delivered. Additionally, the method may further comprise preventing delivery of another drug bolus during a cool down period following the determination that the second portion of the drug bolus has been delivered and/or sending instructions to the drug delivery device indieating that another drug bolus shall not be delivered within the cool down period following the determination that the second portion of the drug bolus has been delivered. The method may further comprise canceling the auto bolus capability if an activity mode is set wherein the activity mode being set indicates that the user is active or going to be active shortly or if the user is in the cool down period. The method may further cause the processor to cancel the relaxing of the at least one drug delivery constraint if at least one cancelation condition other than the expiration of the period is satisfied. The cancelation condition(s) may include at least one of a difference between consecutively received blood glucose values for the user that exceeds a threshold or an activity mode is set wherein the activity mode being set indicates that the user is active or going to be active shortly.

In accordance with another inventive facet, a drug delivery system for delivery of drug to a user may include a storage or reservoir of drug such as insulin and a needle and/or cannula for piercing the skin of the user to deliver the drug from the storage. The drug delivery system may further include a fluid path for the drug between the storage to the needle or cannula. The drug delivery system may include a non-transitory computer-readable storage medium storing historical information and computer programming instructions and a processor configured to execute the computer programming instructions. Executing the computer programming instructions may cause the processor to constrain delivery of the drug to the user per current drug safety constraints and to receive an indication of a user request to deliver a drug bolus immediately to the user. The executing of the computer programming instructions may further cause the processor to, responsive to the received request, deliver a first portion of the drug bolus to the user, and relax at least one of the current drug safety constraints for a period following the delivery of the first portion of the drug bolus so that larger doses of basal drug deliveries may be delivered if needed without being subject to the at least one of the current drug safety constraints that were relaxed.

The drug delivery system may include an element that may be activated by the user to request delivery of the drug bolus. The element may be, for example, one of a button, a knob, a switch, a lever, or a user interface element. The executing of the computer programming instructions may further cause the processor to deliver a second portion of the drug bolus. The executing of the computer programming instructions may further cause the processor to determine a dose for the second portion of the drug bolus. The executing of the computer programming instructions may further cause the processor to prevent delivery of another drug bolus during a cool down period following delivery of the second portion of the drug bolus. The executing of the computer programming instructions may further cause the processor to cancel the relaxing of the at least one drug safety constraint if differences between consecutively received blood glucose values for the user exceed respective thresholds or if an activity mode is set wherein the activity mode being set indicates that the user is active or going to be active shortly.

In accordance with an additional inventive facet, a method configured to be executed on or performed by a processor of a drug delivery system may comprise receiving an indication of a user request to deliver a drug bolus immediately to the user. The method may further comprise responsive to the received request, determine a first portion of the drug bolus to be delivered the user, determining that the first portion of the drug bolus has been delivered by the drug delivery system and relax at least one of a current drug safety constraint for a period following the delivery of the first portion of the drug bolus, wherein relaxing the at least one drug safety constraint allows determination of larger doses of basal drug deliveries to be delivered if needed without being subject to the at least one drug safety constraints that were relaxed.

In accordance with an additional inventive facet, a drug delivery device includes a drug reservoir storing a drug such as insulin and a needle and/or cannula for piercing skin of a patient, said needle and/or cannula being hollow so as to serve as a conduit for delivering drug to the user. The drug delivery device may include a non-transitory computer-readable storage storing computer programing instructions for controlling operation of the drug delivery device. The drug delivery device may include a processor for executing the computer programming instructions to cause the processor to constrain delivery of the drug to the user per current drug delivery constraints, deliver a first portion of the drug bolus to the user, and relax at least one of the current drug safety constraints for a period following the delivery of the first portion of the drug bolus so that larger doses of basal drug deliveries may be delivered if needed without being subject to the at least one current drug safety constraints that were relaxed.

In accordance with an additional inventive facet, a method configured to be executed on or performed by a processor of a drug delivery system may comprise determining a first portion of a drug bolus to be delivered the user, determine that the first portion of a drug bolus has been delivered by the drug delivery system and relax at least one of the current drug safety constraints for a period following the delivery of the first portion of the drug bolus, wherein relaxing the at least one drug safety constraint allows determination of larger doses of basal drug deliveries to be delivered if needed without being subject to the at least one drug safety constraints that were relaxed.

Multiple of the current drug safety constraints may be relaxed. The drug safety constraints may include at least one of a maximum amount of drug that can be delivered to the user from the drug delivery device in an operational cycle of the drug delivery device, a maximum amount of drug that can be delivered to the user from the drug delivery device in a specified number of operational cycles of the drug delivery device, a setpoint for glucose or other analyte level of the user, a maximum level of drug on board for the user, and a penalty amount in a cost function for extra drug delivery. The executing of the computer programming instructions may further cause the processor to deliver a second portion of the drug bolus at a fixed time after delivery of the first portion of the drug bolus. The method may further comprise determining a second portion of the drug bolus to be delivered to the user at a fixed time after delivery of the first portion of the drug bolus.

### BRIEF DESCRIPTION OF THE DRAWINGS

FIG. 1 depicts a block diagram of a drug delivery system of exemplary embodiments.

FIG. 2 depicts a flowchart of illustrative steps that may be performed in exemplary embodiments with an AutoBolus capability.

FIG. 3A depicts a flowchart of illustrative steps that may be performed in exemplary embodiments to obtain meal signals for automatic meal detection.

FIG. 3B depicts an example sliding window that slides over time to encompass an updated two-hour period as new glucose level readings are received for meal detection in exemplary embodiments.

FIG. 4 depicts a flowchart of illustrative steps that may be performed in exemplary embodiments to calculate a dose for a portion of the drug bolus.

FIG. 5 depicts a flowchart of illustrative steps that may be performed tin exemplary embodiments to determine safe required drug, such as insulin, on board, or IOB.

FIG. 6 depicts example of safety constraints that may be relaxed in exemplary embodiments.

FIG. 7 depicts an illustrative plot for an exemplary AutoBolus delivery in exemplary embodiments.

FIG. 8 depicts a flowchart of illustrative steps that may be performed in exemplary embodiments in capping drug bolus portion doses.

FIG. 9 depicts a flowchart of illustrative steps that may be performed in exemplary embodiments to choose among doses for a portion of an drug bolus.

FIG. 10 depicts a flowchart of illustrative steps that may be performed in exemplary embodiments for a cool down period.

FIG. 11 depicts a flowchart of illustrative steps that may be performed in exemplary embodiments to halt the AutoBolus capability.

FIG. 12 depicts a flowchart of illustrative steps that may be performed in exemplary embodiments to cancel the relaxation of the one or more safety constraints.

FIG. 13 depicts a couple of illustrative cancelation conditions that may be used in exemplary embodiments.

FIG. 14A depicts an example of a user interface button on a management device that may be activated by a user to announce eating a meal.

FIG. 14B depicts an illustrative drug delivery device of an exemplary embodiment that has a button on its housing that may be depressed by a user to announce eating a meal.

FIG. 14C depicts a number of illustrative activatable elements that may be activated by a user to announce eating a meal.

FIG. 15 depicts a flowchart of illustrative steps that may be performed in exemplary embodiments to deliver a meal drug bolus and relax at least one safety restraint responsive to activation by a user of an activatable element to announce eating a meal.

FIGS. 16A, 16B, and 16C depict plots that illustrate cooperation of the AutoBolus capability, and the manual meal announcement induced meal drug bolus capability of exemplary embodiments.

### DETAILED DESCRIPTION

The exemplary embodiments may reduce the burden on diabetic patients regarding delivery of meal boluses of drug such as insulin. The exemplary embodiments may provide a drug delivery device and/or drug delivery system that receives glucose and/or other analyte level values for a user (e.g., a diabetic patient or person with diabetes (PWD)) and based on the glucose or other analyte level values, determine when the user has consumed a meal. In some embodiments, the drug delivery device and/or drug delivery system may calculate an appropriate bolus dose and automatically deliver the drug bolus to the user. Thus, the user is relieved of the burden of remembering to deliver a drug bolus for a meal and also is relieved of correctly determining the drug bolus dose. In some embodiments, instead of detecting the meal, the user may announce the meal, such as by activating an element on the drug delivery device or on a management device for the drug delivery device, e.g, wherein the management device is part of the drug delivery system. Responsive to the meal announcement, the drug delivery device may calculate the drug bolus dose and deliver the drug bolus.

In conjunction with the delivery of the drug bolus, the drug delivery device may relax safety constraints for a relaxation period following the drug bolus delivery so that additional basal drug may be delivered if needed. The drug bolus dose may be chosen to be conservative (i.e., a dose that may be less than needed to fully respond to the meal consumption) to reduce the risk of the user becoming hypoglycemic. The drug delivery device may rely upon the relaxed safety constraints to enable more aggressive basal drug delivery to complete the compensation needed to bring glucose levels of the user into a desirable range following meal consumption. The relaxation period may be canceled if glucose level trends indicate a substantial decreasing trend or if the user enters a mode indicating that the user is active, such as exercising. In some embodiments, relaxing the safety constraints allows the drug delivery system or device to deliver a higher amount of basal insulin.

The exemplary embodiments may provide a cool down phase wherein an additional drug bolus may not be delivered in a period following delivery of a drug bolus despite a meal being detected or the user announcing a meal.

The exemplary embodiments may accommodate both the automatic bolus delivery responsive to meal detection with safety constraint relaxation and the bolus delivery responsive to a user meal announcement with safety constraint relaxation. The exemplary embodiments may provide measures to resolve conflicts that may arise with the automatic bolus delivery and meal announcement. The drug delivery device may intelligently halt relaxation of the safety constraints and/or meal detection as needed.

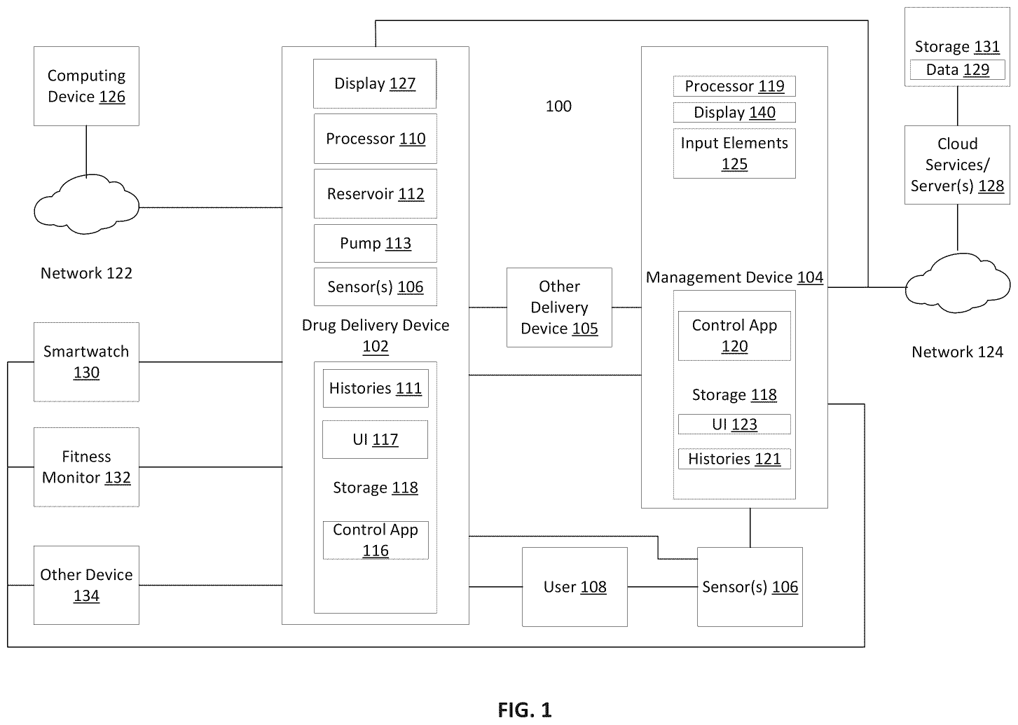

FIG. 1 depicts a block diagram of an illustrative drug delivery system 100 that is suitable for delivering a drug such as insulin to a user 108 in accordance with the exemplary embodiments. The drug delivery system 100 includes a drug delivery device 102. The drug delivery device 102 may be a wearable device that is worn on the body of the user 108 or carried by the user. The drug delivery device 102 may be directly coupled to a user (e.g., directly attached to a body part and/or skin of the user 108 via an adhesive or the like) with no tubes and an infusion location directly under the drug delivery device 102, or carried by the user (e.g., on a belt or in a pocket) with the drug delivery device 102 connected to an infusion site where the drug is injected using a needle and/or cannula. A surface of the drug delivery device 102 may include an adhesive to facilitate attachment to the user 108.

The drug delivery device 102 may include a processor 110. The processor 110 may be, for example, a microprocessor, a logic circuit, a field programmable gate array (FPGA), an application specific integrated circuit (ASIC) or a microcontroller. The processor 110 may maintain a date and time as well as other functions (e.g., calculations or the like). The processor 110 may be operable to execute a control application 116 encoded in computer programming instructions stored in the storage 114 that enables the processor 110 to direct operation of the drug delivery device 102. The control application 116 may be a single program, multiple programs, modules, libraries or the like. The processor 110 also may execute computer programming instructions stored in the storage 114 for a user interface (UI) 117 that may include one or more display screens shown on display 127. The display 127 may display information to the user 108 and, in some instances, may receive input from the user 108, such as when the display 127 is a touchscreen.

The control application 116 may control delivery of the drug to the user 108 per a control approach like that described herein. In exemplary embodiments, the control application 116 may control the termination of the electric pulse to an SMA (Shape Memory Alloy) element as described below. The storage 114 may hold histories 111 for a user, such as a history of basal deliveries, a history of bolus deliveries, and/or other histories, such as a meal event history, exercise event history, glucose level history, other analyte level history, and/or the like. In addition, the processor 110 may be operable to receive data or information. The storage 114 may include both primary memory and secondary memory. The storage 114 may include random access memory (RAM), read only memory (ROM), optical storage, magnetic storage, removable storage media, solid state storage or the like.

The drug delivery device 102 may include a tray or cradle and/or one or more housings for housing its various components including a pump 113, a power source (not shown), and a reservoir 112 for storing drug for delivery to the user 108. A fluid path to the user 108 may be provided, and the drug delivery device 102 may expel the drug from the reservoir 112 to deliver the drug to the user 108 using the pump 113 via the fluid path. The fluid path may, for example, include tubing coupling the drug delivery device 102 to the user 108 (e.g., tubing coupling a cannula to the reservoir 112), and may include a conduit to a separate infusion site. The drug delivery device 102 may have operational cycles, such as every 5 minutes, in which basal doses of drug are calculated and delivered as needed. These steps are repeated for each cycle.

There may be one or more communications links with one or more devices physically separated from the drug delivery device 102 including, for example, a management device 104 of the user and/or a caregiver of the user, sensor(s) 106, a smartwatch 130, a fitness monitor 132 and/or another variety of device 134. The communication links may include any wired or wireless communication links operating according to any known communications protocol or standard, such as Bluetooth®, Wi-Fi, a near-field communication standard, a cellular standard, or any other wireless protocol.

The drug delivery device 102 may interface with a network 122 via a wired or wireless communications link. The network 122 may include a local area network (LAN), a wide area network (WAN), a cellular network, a Wi-Fi network, a near field communication network, or a combination thereof. A computing device 126 may be interfaced with the network 122, and the computing device may communicate with the drug delivery device 102.

The drug delivery system 100 may include one or more sensor(s) 106 for sensing the levels of one or more analytes. The sensor(s) 106 may be coupled to the user 108 by, for example, adhesive or the like and may provide information or data on one or more medical conditions and/or physical attributes of the user 108. The sensor(s) 106 may be physically separate from the drug delivery device 102 or may be an integrated component thereof. The sensor(s) 106 may include, for example, glucose monitors, such as continuous glucose monitors (CGM's) and/or non-invasive glucose monitors. The sensor(s) 106 may include ketone sensors, other analyte sensors, heart rate monitors, breathing rate monitors, motion sensors, temperature sensors, perspiration sensors, blood pressure sensors, alcohol sensors, or the like. Some sensors 106 may also detect characteristics of components of the drug delivery device 102. For instance, the sensors 106 in the drug delivery device may include voltage sensors, current sensors, temperature sensors and the like.

The drug delivery system 100 may or may not also include a management device 104. In some embodiments, no management device is needed as the drug delivery device 102 may manage itself. The management device 104 may be a special purpose device, such as a dedicated personal diabetes manager (PDM) device. The management device 104 may be a programmed general-purpose device, such as any portable electronic device including, for example, a dedicated controller, such as a processor, a micro-controller, or the like. The management device 104 may be used to program or adjust operation of the drug delivery device 102 and/or the sensor(s) 106. The management device 104 may be any portable electronic device including, for example, a dedicated device, a smartphone, a smartwatch, or a tablet. In the depicted example, the management device 104 may include a processor 119 and a storage 118. The processor 119 may execute processes to manage a user's glucose levels and to control the delivery of the drug to the user 108. The drug delivery device 102 may provide data from the sensors 106 and other data to the management device 104. The data may be stored in the storage 118. The processor 119 may also be operable to execute programming code stored in the storage 118. For example, the storage 118 may be operable to store one or more control applications 120 for execution by the processor 119. Storage 118 may also be operable to store historical information such as drug delivery information, analyte level information, user input information, output information, or other historical information. The control application 120 may be responsible for controlling the drug delivery device 102, such as by controlling the automated drug delivery (ADD) (or, for example, automated insulin delivery (AID)) of drug to the user 108. In some exemplary embodiments, the control application 120 provides the adaptability described herein. The storage 118 may store the control application 120, histories 121 like those described above for the drug delivery device 102, and other data and/or programs. The embodiments presented herein may also be performed by a plurality of processors for example in a distributed computer system.

A display 140, such as a touchscreen, may be provided for displaying information. The display 140 may display user interface (UI) 123. The display 140 also may be used to receive input, such as when it is a touchscreen. The management device 104 may further include input elements 125, such as a keyboard, button, knobs, or the like, for receiving input form the user 108.

The management device 104 may interface with a network 124, such as a LAN or WAN or combination of such networks, via wired or wireless communication links. The management device 104 may communicate over network 124 with one or more servers or cloud services 128. Data, such as sensor values, may be sent, in some embodiments, for storage and processing from the drug delivery device 102 directly to the cloud services/server(s) 128 or instead from the management device 104 to the cloud services/server(s) 128.

Other devices, like smartwatch 130, fitness monitor 132 and device 134 may be part of the drug delivery system 100. These devices 130, 132 and 134 may communicate with the drug delivery device 102 and/or management device 104 to receive information and/or issue commands to the drug delivery device 102. These devices 130, 132 and 134 may execute computer programming instructions to perform some of the control functions otherwise performed by processor 110 or processor 119, such as via control applications 116 and 120. These devices 130, 132 and 134 may include displays for displaying information. The displays may show a user interface for providing input by the user, such as to request a change or pause in dosage, or to request, initiate, or confirm delivery of a bolus of drug, or for displaying output, such as a change in dosage (e.g., of a basal delivery amount) as determined by processor 110 or management device 104. These devices 130, 132 and 134 may also have wireless communication connections with the sensor 106 to directly receive analyte measurement data. Another delivery device 105, such as a drug delivery pen (such as an insulin pen), may be accounted for (e.g., in determining IOB) or may be provided for also delivering drug to the user 108.

The functionality described herein for the exemplary embodiments may be under the control of or performed by the control application 116 of the drug delivery device 102 or the control application 120 of the management device 104. In some embodiments, the functionality wholly or partially may be under the control of or performed by the cloud services/servers 128, the computing device 126 or by the other enumerated devices, including smartwatch 130, fitness monitor 132 or another wearable device 134.

In the closed loop mode, the control application 116, 120 determines the drug delivery amount for the user 108 on an ongoing basis based on a feedback loop. For a drug delivery device that uses insulin, for example, the aim of the closed loop mode is to have the user's glucose level at a target glucose level or within a target glucose range. In some embodiments, the target glucose level is between about 100 mg/dL to about 140 mg/dL, more specifically between about 110 mg/dL to about 130 mg/dL and in particular between about 115 mg/dL to about 120 mg/dL.

In some embodiments, the drug delivery device 102 need not deliver one drug alone. Instead, the drug delivery device 102 may one drug, such as insulin, for lowering glucose levels of the user 108 and also deliver another drug, such as glucagon, for raising glucose levels of the user 108. The drug delivery device 102 may deliver a glucagon-like peptide (GLP)-1 receptor agonist drug for lowering glucose concentration in the blood or slowing gastric emptying, thereby delaying spikes in glucose after a meal. In other embodiments, the drug delivery device 102 may deliver pramlintide, or other drugs that may substitute for insulin. In other embodiments, the drug delivery device 102 may deliver concentrated insulin. In some embodiments, the medicament or drug delivered by the drug delivery device may be a coformulation of two or more of those medicaments identified above. In a preferred embodiment, the drug delivery device delivers insulin; accordingly, reference will be made throughout this application to insulin and an insulin delivery device, but one of ordinary skill in the art would understand that drugs other than insulin can be delivered in lieu of or in addition to insulin.

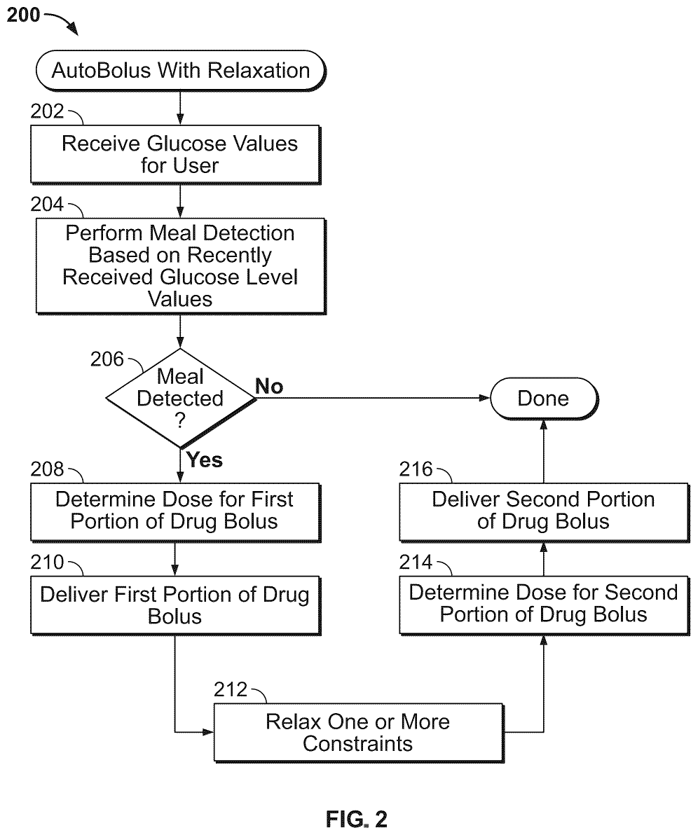

As mentioned above, the exemplary embodiments may automatically detect meal ingestion by a user 108 based on glucose level values, and automatically deliver an insulin bolus to offset the rise in glucose due to the ingestion of the meal. The automatic delivery of the bolus may be referred to herein as “AutoBolus.” FIG. 2 depicts a flowchart 200 of illustrative steps that may be performed in exemplary embodiments in providing the AutoBolus capability. At 202, glucose level values are received for the user. The glucose level values may originate from a glucose sensor, such as a CGM, that is one of the sensor(s) 106. Where the glucose level values originate from a CGM, the glucose values may be received via a wireless connection between the CGM and the drug delivery device 102. The glucose level values may be sent through an intermediate device, like the management device 104, on their way to the drug delivery device 102. Moreover, the glucose level values may be stored in storage 118 and retrieved from the storage 118 as needed. The glucose level values may be the most recent ones obtained for the user 100 and may include enough values to detect meals as is explained below (e.g., glucose level values for rolling 15 minute windows). Alternatively, the glucose level values may be obtained from a glucometer that reads test strips on which a drop of the user's blood has been applied or from another variety of glucose sensor. The most recent glucose level value may be the glucose level values received closest to the current time, e.g, wherein the current time is the point in time where the automatic meal detection takes place.

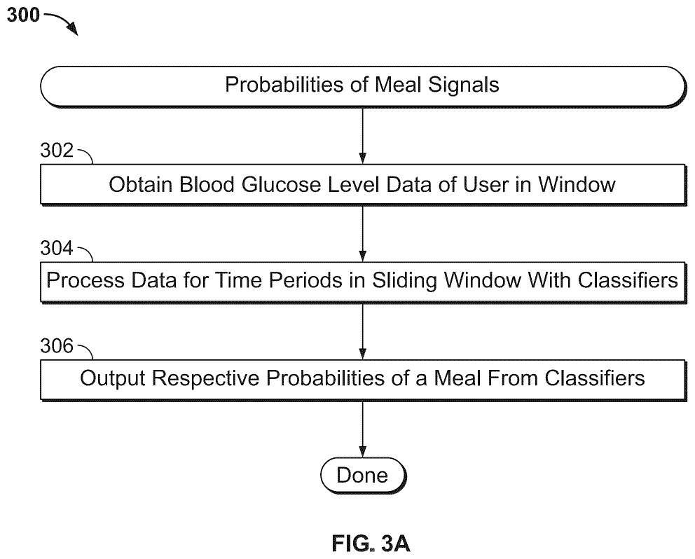

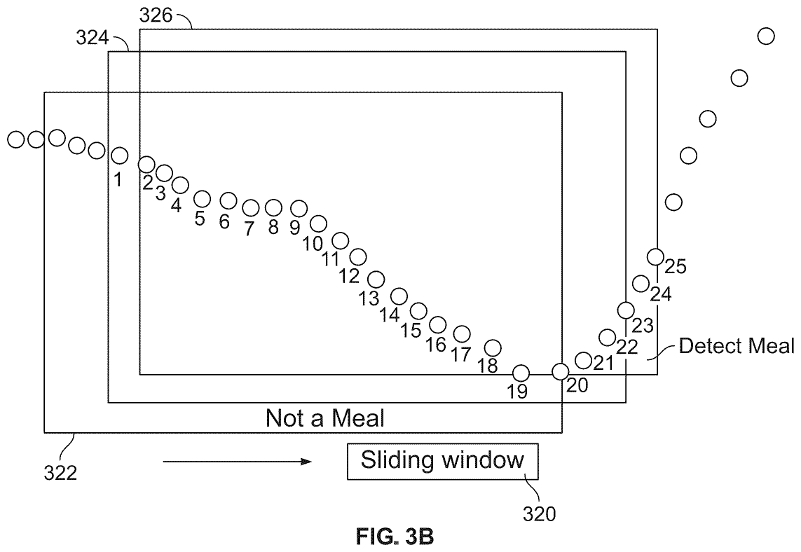

At 204, the glucose level values are processed to detect whether the user 108 has ingested a meal. The exemplary embodiments may gather meal signals based on glucose level values from the user over a time window and use the meal signals to determine if a meal has been detected or not at 203. The meal signals represent probabilities that a meal has been detected in glucose level values for the user 108 at successive time intervals. FIG. 3A depicts a flowchart 300 of illustrative steps that may be performed in exemplary embodiments to obtain the meal signals. In accordance with this approach during training of the machine learning model for meal detection, glucose rises of specified or predetermined magnitudes within respective time periods are sought. For example, the approach may look for instances of a 20 mg/dL rise or greater within a 15-minute period, a 40 mg/dL rise or greater in a 30-minute period and a 60 mg/dL rise or greater in a 60-minute period. As an embodiment in model prediction mode, limited data from 10 minutes, 15 minutes and 20 minutes of glucose values are used to predict if the glucose is going to rise in the next 30 minutes to 60 minutes time frame. At 302, blood glucose level data of the user 108 is obtained for 10-minute periods, 15-minute periods and 20-minute periods within a window, such as a two-hour window, ending with a most recent glucose level reading. In some embodiments, the glucose values of the prior about 5 minutes to about 40 minutes (relative to the current time), more specifically the glucose values of the prior about 10 minutes to about 30 minutes and in particular of the prior 10 minutes to about 20 minutes are used to predict if the glucose is going to rise in the next 30 minutes to 60 minutes time frame.

In the exemplary embodiments, at 304, classifiers may process the glucose level values to predict glucose level rises using glucose values in 10 minute, 15 minute and 20 minute time windows. A separate classifier may be provided for each time period. The classifiers may be machine learning models that recognize patterns of glucose level rises indicative of meals. The classifiers may be part of the control application 116 or 120. One classifier may utilize glucose values in a 10-minute interval in the window. Another classifier may utilize glucose values in a 15-minute interval in the window, and a third classifier may utilize glucose values in a 20 minute interval in the window. Each classifier may be, for example, a separate neural network model or a separate decision tree model. The classifiers may output the probability that a meal has been detected within the window based on computed features in the time window such as first and second derivative, mean values, range values in the window, etc. While the 10 minute, 15 minute and 20 minute time windows for predicting glucose rise probabilities are used as exemplary embodiments, it should be noted that larger windows that utilize more glucose values for example, 30 minute, 60 minute, 120 minute windows are other possible embodiments, for example.

At 306, the classifiers may output their respective probabilities, which are used in determining probabilities of whether there has been a meal event, and in determining the maximum allowed insulin bolus dose, as described above. The monitoring of the glucose level data of the user 108 may be performed on an ongoing basis.

FIG. 3B depicts an example sliding window 320 that slides over time to encompass an updated two-hour period as new glucose level readings are received. The sliding window 320 may start off in position or window 322 that covers glucose level readings up to reading number 20. In a second position or window 324, the window encompasses readings from reading 1 to reading 23, and in the third position or window 326, the window encompasses from reading 2 to reading 25. Each reading may occur at 5-minute intervals; thus, one window may encompass a two-hour period. In positions of windows or positions 322 and 324, no meal is detected, but a meal is detected in position or window 326 based on the predicted glucose level rise.

With reference to FIG. 2, at 206, a check may be made whether a meal is detected based on the glucose level values. If not, the process of FIG. 2 is complete. If so, at 208, a dose for a first portion of the insulin bolus may be determined. In the exemplary embodiments, a first portion of the insulin bolus may be delivered by the AutoBolus mechanism immediately after the meal is detected. This may be followed by the delivery of a second portion of the insulin bolus, such as during a next operational cycle, as is explained below. The dose for the first portion of the insulin bolus may be chosen to be a safe amount to be delivered immediately.

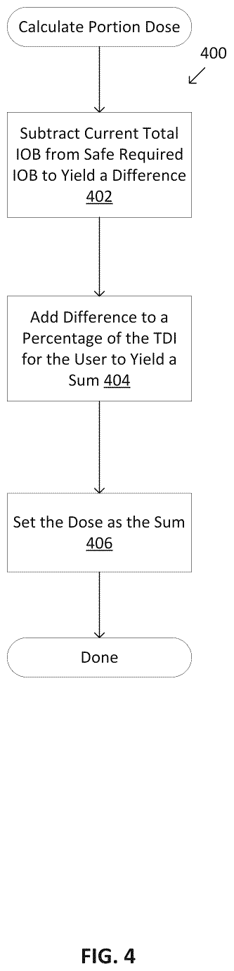

FIG. 4 depicts a flowchart 400 of illustrative steps that may be performed in exemplary embodiments to calculate a dose for a portion of the insulin bolus, in particular for the first portion. At 402, the current total Insulin on Board (IOB) for the user 108 may be subtracted from a safe required IOB to yield a difference. The total IOB includes all of the basal and bolus insulin delivered to the user that still may affect the glucose level of the user. The safe required IOB represents an IOB level that has a safety factor built into it. At 404, the difference may be added to a preset percentage of the total daily insulin (TDI) for the user 108. TDI may be the total of all insulin, basal and bolus, that is delivered to the user 108 in a day. The preset percentage may range, for example, between 3 to 8 percent of TDI. The sum reflects a percentage of TDI plus any extra insulin required to reach the safe required IOB level for the user 108. At 406, the dose for the portion, in particular the first portion, of the insulin bolus may be set as the sum. This may be expressed as:

$$meal dose = x \% T D I + safe Required I O B - Current Total I O B .$$

Accordingly, in some embodiments, the first portion is determined by determining a safe required IOB and determining a current total IOB, and adding the safe required IOB to a fraction of the total daily insulin and subtracting the current Total IOB therefrom. In some embodiments, the fraction of the total daily insulin is calculated as the total daily insulin for the user multiplied by a meal factor, wherein the meal factor is between about 1% to about 10%, more specifically 3% to 8%. Some user's may prefer having at minimum always delivered to feel safer when consuming a meal. Accordingly, in some embodiments, the first portion may be at least between about 0.3% to about 3% of the TDI, more specifically at least between about 0.5% to about 1.5% of the TDI and in particular at least about 0.8% to about 1.2% of the TDI. In some embodiments, the first portion may be at most between about 0.3 Units to about 3 Units, more specifically between about 0.5 Units to about 1.5 Units and in particular between about 0.8 Units to about 1.2 Units of insulin.

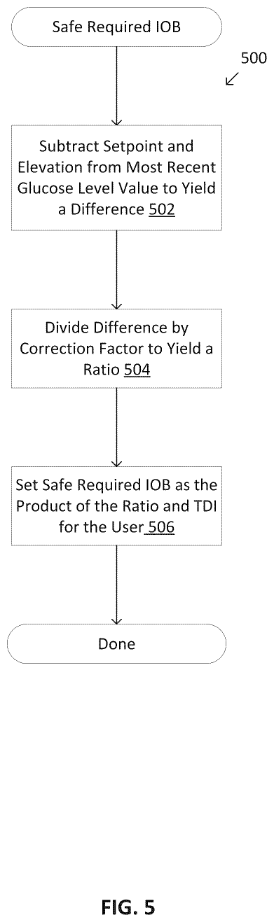

FIG. 5 depicts a flowchart 500 of illustrative steps that may be performed to determine safe required IOB. A suitable equation for calculating the safe required IOB in exemplary embodiments is:

$$Safe Required I O B = ( \frac{Current BG - ( Setpoint + Elevation )}{Correction Factor} ) * T D I$$

and the flowchart 500 depicts steps for calculating the safe required IOB per this formula. Accordingly in some embodiments, the safe required IOB is calculated by calculating a first value as subtracting from the current glucose level value for the user the sum of the setpoint and the elevation. Then, calculating the safe required IOB by calculating a second value by dividing the first value by the user's correction factor and multiplying the second value with the user's TDI. At 502, the setpoint for the drug delivery device 102 and an elevation value are subtracted from the current glucose level value for the user 108. The resulting difference captures the difference between a glucose level value and an elevated value that is a setpoint plus an elevation amount. The setpoint may be, for example, 120 mg/dL, and the elevation may be a value such as 30 mg/dL. In some embodiments, the setpoint is between about 100 mg/dL to about 140 mg/dL, more specifically between about 110 mg/dL to about 130 mg/dL and in particular between about 115 mg/dL to about 120 mg/dL. In some embodiments, the elevation is between about 10 mg/dL to about 50 mg/dL, more specifically between about 20 mg/dL to about 40 mg/dL and in particular between about 25 mg/dL to about 35 mg/dL. The elevation may act a safety factor, which results in a lower insulin delivery to prevent hypoglycemia following insulin delivery. At 504, the difference may be divided by a correction factor to yield a ratio. At 506, the safe required IOB is set as the product of the ratio and the TDI for the user 108.

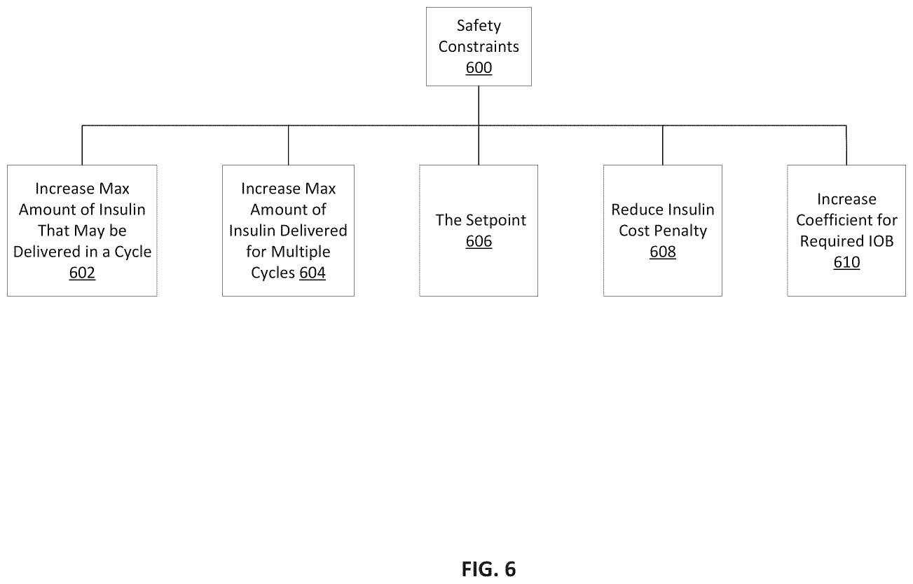

With reference to FIG. 2, at 210, the first portion of the insulin bolus may be delivered to the user 108 by the insulin delivery device 102. At 212, the relaxation of one or more safety constraints may start during the next cycle after meal detection. The relaxation of one or more safety constraints may enable more insulin to be delivered as basal insulin deliveries during a relaxation period. The relaxation period may last, for example, a time frame of 6 cycles. FIG. 6 depicts example of safety constraints that may be relaxed in exemplary embodiments. The maximum amount of insulin that may be delivered in a cycle 602 is a safety constraint that may be relaxed. For instance, the maximum amount of insulin that may be delivered in a cycle may be a multiple of the ideal basal delivery amount per cycle, and the relaxing may cause the multiple to increase. Another safety constraint that may be relaxed is the maximum amount of insulin that may be delivered over multiple cycles 604. The relaxing may simply increase this maximum. The set point 606, also known as the “target,” for the control application 116 or 120 is a safety constraint. The set point 606 may be lowered as part of the relaxing of constraints. For example, the greater of the current setpoint minus 20 or the fixed value of 90 mg/dL may be chosen as the new setpoint in some exemplary embodiments. In embodiments, the fixed value may be between about 70 mg/dL to about 110 mg/dL, more specifically between about 80 mg/dL to about 100 mg/dL and in particular between about 85 mg/dL to about 95 mg/dL. A further safety constraint is the cost component for insulin 608 (i.e., insulin cost). This penalty for excess insulin delivery may be decreased as part of the relaxing. A typical cost function for insulin delivery is:

$$J ( {I}_{rec} ) = {Q ( f ( {I}_{rec} ) - {G}_{target} )}^{n} + {R ( {I}_{rec} - {I}_{b} )}^{m}$$

where J is the total penalty, I_{rec} is the current recommended insulin delivery being assessed for the total penalty, Q is the coefficient of the glucose excursions, ƒ(I_{rec}) is any generic function to associate this recommended insulin delivery with a corresponding expected glucose value, G_{target} is the current control target, R is the coefficient for insulin excursions, I_{b} is the current baseline insulin delivery, and n and m are generic coefficients representing any scaling of the penalties for glucose and/or insulin excursions. The control application 116 or 120 may choose basal doses based on choosing a basal dose with a best (i.e., lowest) cost. The insulin cost is captured by R(I_{rec}−I_{b})^{m}, and the coefficient R may be modified to relax the constraint.

At 214, the steps of FIG. 4 are repeated with an updated total IOB and updated safe required IOB given the new glucose level value and given the first portion of the insulin bolus having been delivered. Once the dose, in particular for the second portion, is determined, the second portion of the insulin bolus may be delivered at 216. In some embodiments, the second portion is determined 1 to 5 cycles, more specifically 1 to 3 cycles after the determination of the first portion, in particular during the cycle directly after the cycle at which the first portion was determined. In some embodiments, the second portion is determined per the same calculation as the first portion, at a cycle following the calculation of the first portion, in particular the cycle following the cycle at which the first portion was determined, wherein the current total IOB, the safe required IOB and the blood glucose value are updated to the cycle following the calculation. Accordingly, in some embodiments, the second portion is determined by determining a safe required IOB (for the cycle at which the second portion is determined) and determining a current total IOB (for the cycle at which the second portion is determined), and adding the safe required IOB (for the cycle at which the second portion is determined) to a fraction of the total daily insulin and subtracting the current Total IOB (for the cycle at which the second portion is determined) therefrom. In some embodiments, the fraction of the total daily insulin is calculated as the total daily insulin for the user multiplied by a meal factor, wherein the meal factor is between about 1% to about 10%, more specifically 3% to 8%.

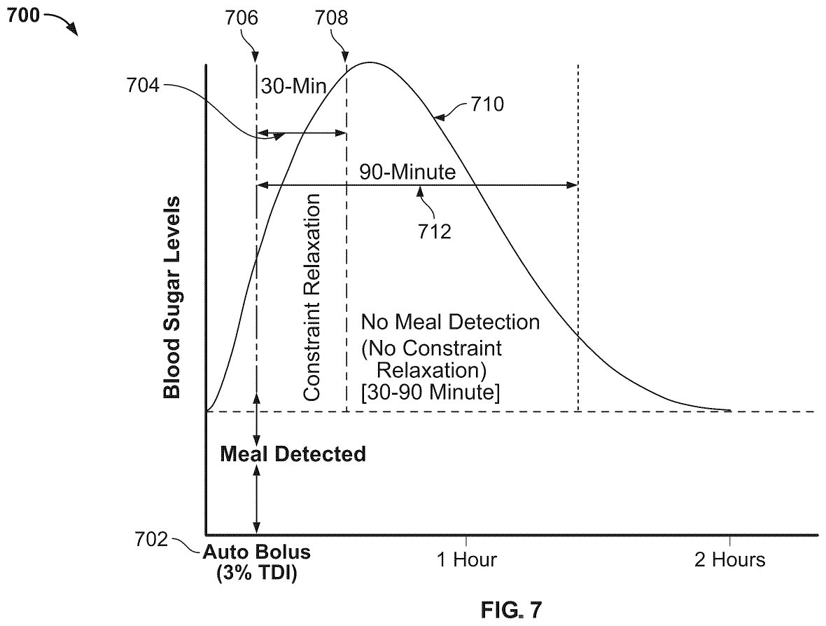

FIG. 7 depicts an illustrative plot 700 for an exemplary AutoBolus delivery. The plot 700 shows that a meal is detected at time 702. The first portion of the insulin bolus may be delivered at that time and may be followed shortly (e.g., the next cycle) by delivery of the second portion. The safety constraints may be relaxed immediately for a 30 minute period 704 between times 706 and 708. The plot 700 also shows a curve 710 of the glucose level values for the user 108 over time. As is discussed below, there may be a cool down period of, for example, 90 minutes as indicated by arrow 712 after the insulin bolus is delivered. During the cool down period, there may be no meal detection so no additional boluses can be automatically delivered via the AutoBolus mechanism.

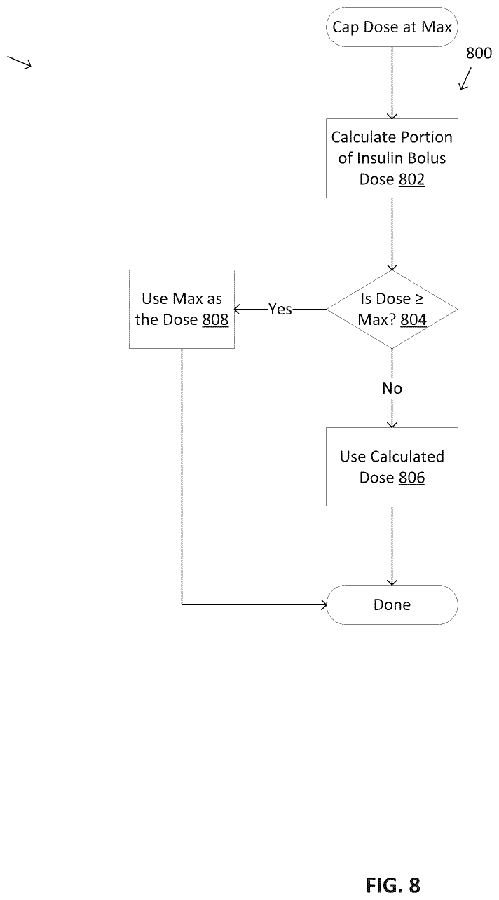

There are alternatives for determining the doses for the portions of the insulin bolus that differ from the approach discussed above. In some exemplary embodiments the dose for each portion of the insulin bolus may be capped. FIG. 8 depicts a flowchart 800 of illustrative steps that may be performed in exemplary embodiments in capping the doses. At 802, the dose for the portion of insulin bolus may be calculated such as described above. The dose may be compared to a maximum, such as 1 unit of insulin, that serves as a cap at 804. If the dose is greater than or equal to the cap, then the maximum may be used as the dose that ultimately may be delivered at 808. If not, the calculated dose may be used as the dose that may be delivered.

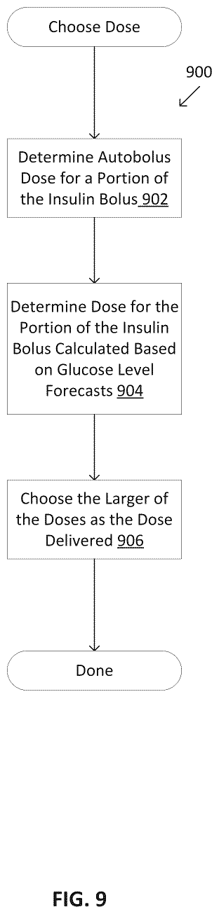

In other exemplary embodiments a dose is chosen among options. FIG. 9 depicts a flowchart 900 of illustrative steps that may be performed in exemplary embodiments to choose among options. At 902, the dose may be determined for the AutoBolus delivery as described above relative to FIG. 4. At 902, a dose may be determined for the portion of the insulin bolus based on glucose level forecasts. For instance, the insulin delivery device 102 may include logic in the control application 116 that determines what bolus dose is needed to bring the glucose level of the user 108 into an acceptable range given the current glucose level of the user 108, the meal consumption and glucose level trends. At 906, the larger of the two determined doses may be chosen for the portion of the insulin bolus. It is presumed that both dose sizes are safe, and hence, the larger dose may be chosen to more quickly bring the glucose level values of the user 108 into an acceptable range.

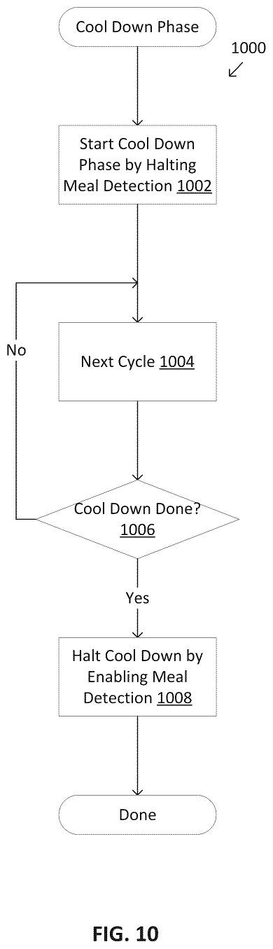

As was mentioned above, a cool down period may be provided. The cool down period helps to prevent excessive bolusing that may cause the glucose level of the user 108 to crash or to reach hypoglycemic levels or more generally, undesirably low levels. In some embodiments, the hypoglycemic level may be defined as blood glucose level between about between about 40 mg/dL to about 80 mg/dL, more specifically between about 50 mg/dL to about 70 mg/dL and in particular between about 55 mg/dL to about 65 mg/dL. The cool down period may be a fixed number of cycles, such as 12-18 cycles (i.e., with 5 minute cycles, the cool down period lasts 1 hour to 1.5 hours). In some embodiments, the cool down period has fixed number of cycles, wherein the number of cycles is between about 3 cycles to about 180 cycles, more specifically 6 cycles to about 60 cycles, and in particular between about 10 cycles to about 20 about cycles. In some embodiments, each cycle has a length between about 30 seconds to about 30 minutes, more specifically between about 1.5 minutes to about 10 minutes and in particular between about 3 minutes to about 9 minutes. FIG. 10 depicts a flowchart 1000 of illustrative steps that may be performed in exemplary embodiments for the cool down period. At 1002, the cooldown period may be entered due to delivery of an insulin bolus. The cool down period may be realized by halting meal detection and thus, halting any AutoBolus insulin deliveries. At 1004, time progresses until the next cycle is reached. At 1006, a check may be made whether the cool down period is done. If not, the process may repeat waiting until the next cycle is reached at 1004. If the end of the cool down period is reached, at 1008, the cool down period may be halted by enabling meal detection. It should be appreciated that means other than turning on and off meal detection may be used to enable and disable the AutoBolus capability.

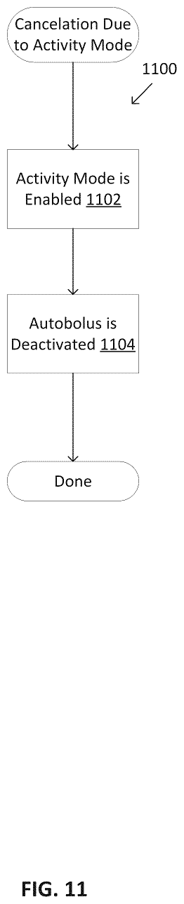

The AutoBolus capability may also be deactivated when the user exercises to prevent the user from experiencing excessively low glucose levels. FIG. 11 depicts a flowchart 1100 of illustrative steps that may be performed in exemplary embodiments to halt AutoBolus in such instances. The insulin delivery device 102 may have an activity mode that the user 108 may select when the user 108 begins exercising or is about to exercise. At 1102, the user 108 turns on the activity mode. In some embodiments, the insulin delivery device 102 automatically detects activity, such as exercise, and triggers the activity mode. At 1106, the AutoBolus capability may be then deactivated, such as described above. The AutoBolus capability may be reactivated, in some embodiments, responsive to the activity mode being turned off.

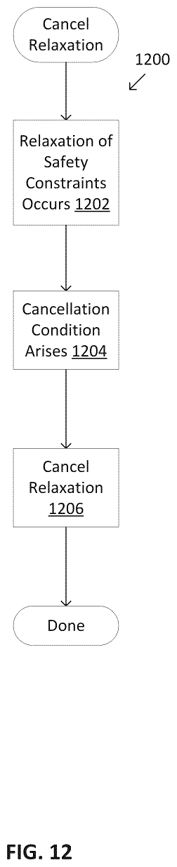

As was mentioned above, one or more safety constraints may be relaxed responsive to the delivery of the insulin bolus. This relaxation, however, may be canceled in some exemplary embodiments if cancelation conditions arise. FIG. 12 depicts a flowchart 1200 of illustrative steps that may be performed in exemplary embodiments to cancel the relaxation of the one or more safety constraints. At 1202, the safety constraints are relaxed responsive to delivery of an insulin bolus. At 1204, a cancelation condition arises. At 1206, the relaxation of the one or more safety constraints may be canceled so that the one or more safety constraints revert to default settings or to most recently established values prior to the relaxation.

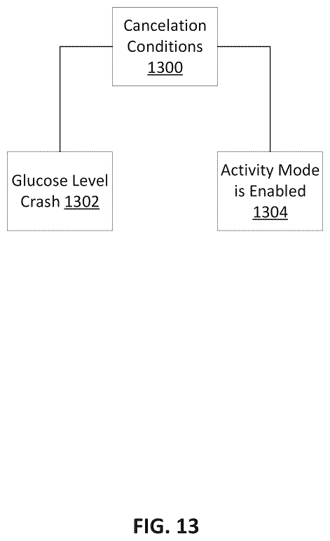

The cancelation conditions may vary. FIG. 13 depicts a couple of illustrative cancelation conditions 1300. A glucose level crash 1302 may trigger cancelation of the relaxation so as to reduce the basal insulin delivery and allow the glucose level of the user 108 to stabilize. A glucose level crash 1302 may be specified as, for example, the glucose level falling below a threshold or the glucose level of the user 108 to be dropping at more than a specified threshold rate. In some embodiments, the rate of change threshold rate may be between about −1 mg/dL/min to about −10 mg/dL/min, more specifically between about −2 mg/dL/min to about −6 mg/dL/min and in particular −2.5 mg/dL/min to about −4 mg/dL/min. Alternatively or additionally, in some embodiment a glucose level crash 1302 may be specified as the glucose level falling for at least three cycles. In some embodiments, the glucose level crash may be specified if the rate of change threshold rate is exceeded between two subsequent cycles and/or between three subsequent cycles. In some embodiments, a glucose level crash may be determined if the of change threshold rate is exceeded between two subsequent cycles or three subsequent cycles, wherein the rate of change threshold for the three subsequent cycles is lower when determining a glucose level crash over three subsequent cycles compared too over two subsequent cycles, more specifically between about 20% to about 70% lower, in particular between about 40% to about 60% lower. In some embodiments, a glucose level crash may be specified if the glucose level decreases between four subsequent cycles. Another cancelation condition may be that an activity mode is enabled 1304. The exercise performed by the user when in activity mode, or in other words when the user is exercising, will cause the glucose level of the user to drop 108, so there is a risk that the potential greater insulin delivery due to the relaxed safety constraints may cause the glucose level of the user to drop to too low of a level. Hence, cancelation of the relaxation is warranted.

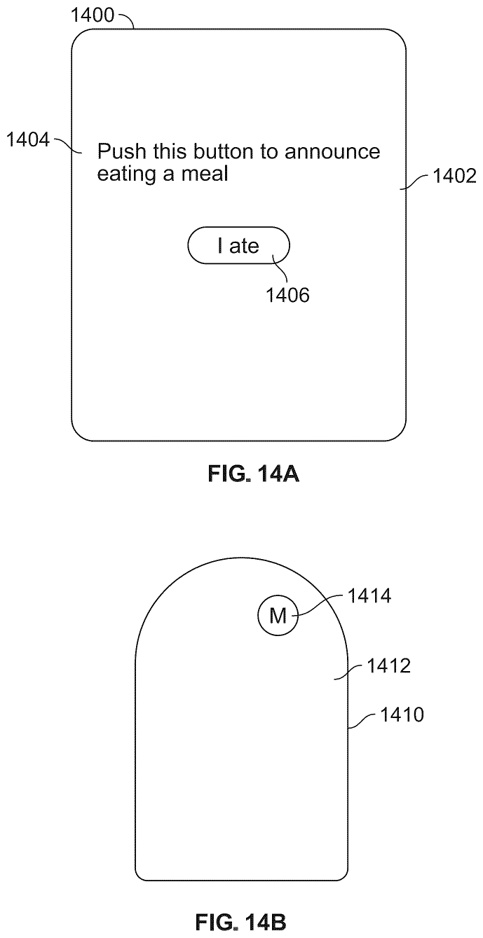

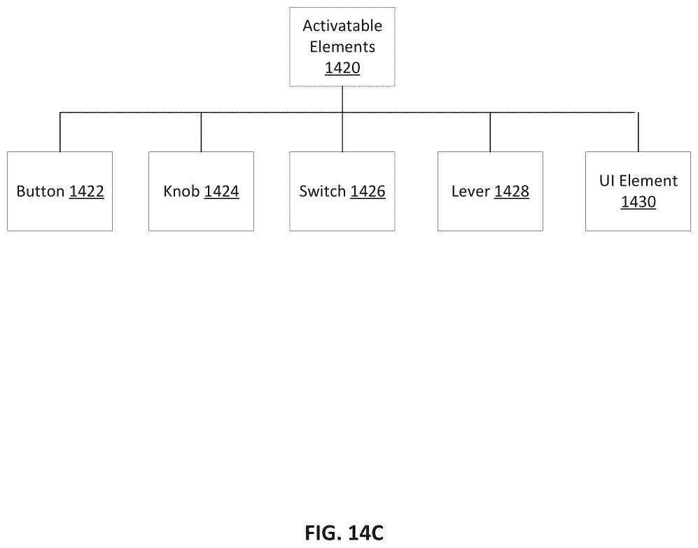

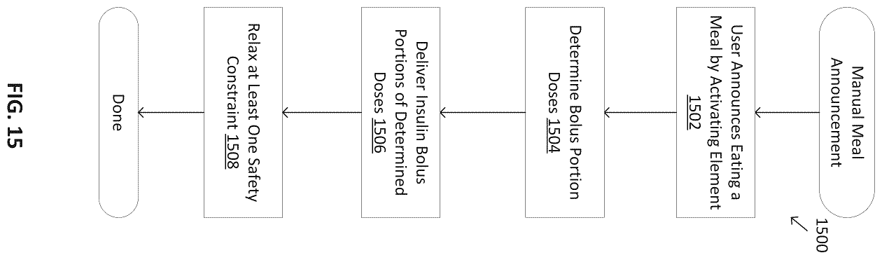

As was mentioned above, the delivery of an insulin bolus and the relaxation of safety constraints for a period may also be triggered by the user 108 activating an element on the drug delivery device 102 or the management device to announce to a meal. For example, as shown in FIG. 14A, the management device 1400 may include a display 1402. The display 1402 may show text that instructs the user 108 to push a button 1406 to indicate the user 108 has eaten a meal. The pushing of the button 1406 announces that the user 108 has eaten a meal and the drug delivery device may deliver a meal insulin bolus as explained below. The element that is activated to announce a meal may instead or in additionally be on the drug delivery device 102. FIG. 14B depicts a top view of a drug delivery device 1410. The top portion of the housing 1412 may include a mechanical, electromechanical, or electrical button 1414 (e.g., a physical or alternatively a “soft” button may be used) that may be depressed or touched to announce that the user has eaten a meal or is about to eat a meal. Settings in drug delivery device 102 may include user-adjustable settings that indicate an approximate time of when a meal will be consumed after pressing button 1414. For example, a user may set the settings to “5-minutes” meaning that the user will typically cat 5-minutes after pressing button 1414. Other types of elements may be provided for the user 108 to announce eating a meal in some embodiments. FIG. 14C depicts a diagram listing some of the types of elements that may be activated to announce that the user 108 has eaten a meal. The activatable elements 1420 may include a button 1422, a knob 1424, a switch 1426, a lever 1428, or a user interface (UI) element 1430. It should be appreciated that other varieties of activatable elements may be used and that the depiction in FIG. 14C is not intended to be exhaustive.

FIG. 15 depicts a flowchart 1500 of illustrative steps that may be performed in exemplary embodiments when the manual meal announcement approach is used. At 1502, the user 108 announces eating a meal by activating the activatable element. In response to the activation, at 1504, the drug delivery device 102 may determine the doses for portions of the insulin bolus that is to be delivered as a meal insulin bolus. This may be done as discussed above relative to FIG. 4. At 1506, the drug delivery device 102 delivers the portions of the insulin bolus as described above relative to FIG. 3. At 1508, at least one of the safety constraints is relaxed as described above.

There may be a cool down period once the portions of the insulin bolus have been delivered as described above. The relaxation of the safety constraints may be canceled as described above relative to claims 12 and 13.

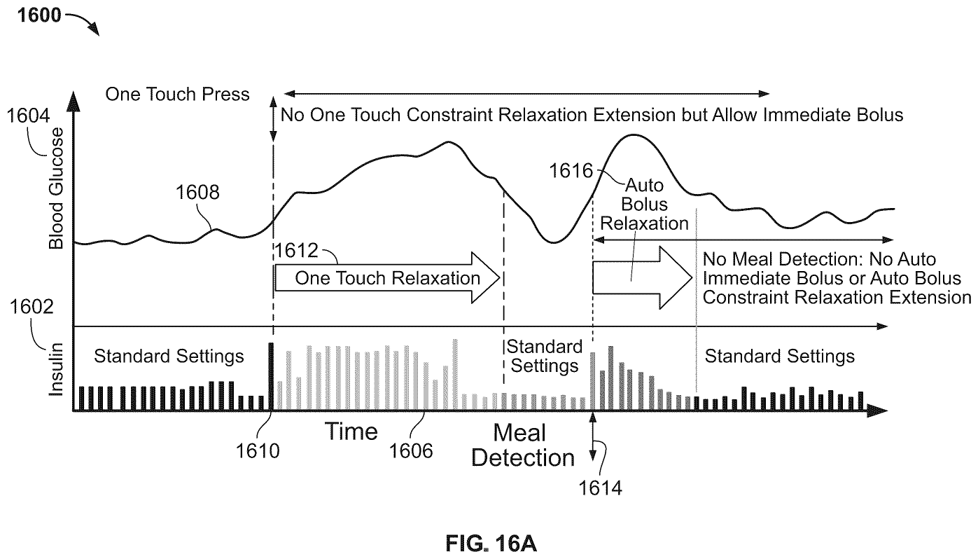

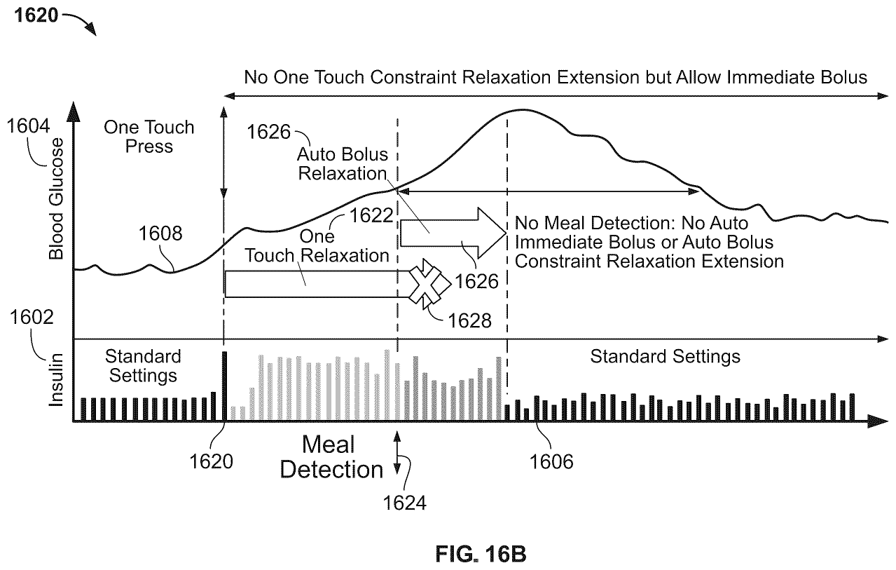

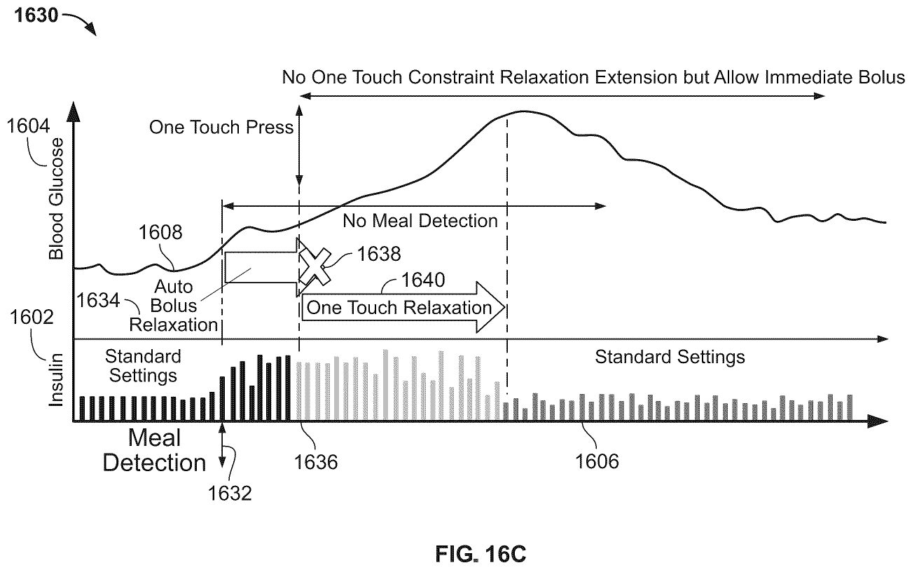

In some exemplary embodiments, the AutoBolus capability and the manual meal announcement may be used together. The drug delivery device 102 may take measures to ensure that these two approaches for identifying meals and delivering meal insulin boluses in response are compatible. FIG. 16A depicts a plot 1600 of operation when the AutoBolus and meal announcement effects do not overlap in their timing. The plot 1600 plots insulin delivery amounts 1602 over time 1606 and glucose levels 1604 of the user over time 1606. Curve 1608 of the glucose levels is shown. The plot 1600 also shows where standard safety settings are in effects as indicated by “standard settings” along the time axis. In this depiction, at time 1610, the user 108 activates an activatable element to announce eating a meal. The drug delivery device 102 determines the doses for a first and second portions of an insulin bolus and delivers the first and second portions of the insulin bolus as described above. At least one of the safety constraints is relaxed from the “standard settings” for the relaxation period as indicated by arrow 1612. After relaxation period ends the safety constraints are no longer relaxed. A meal is detected at time 1614. The drug delivery device determines doses for the first and second portions of an insulin bolus and delivers them to the user 108. At least one safety constraint is relaxed for the relaxation period as indicated by arrow 1616. Once the relaxation period ends, the one or more safety constraints that were relaxed are no longer relaxed.

FIG. 16B depicts a plot 1620 when the AutoBolus capability is triggered while the at least one safety constraint is relaxed from the “standard settings” following insulin bolus delivery responsive to a manual meal announcement. In the depicted instance, at time 1620, the user 108 announces a meal by activating an activatable element. The drug delivery device 102, determines doses for first and second portions of a meal insulin bolus and delivers the first and second portions to the user 108. At least one safety constraint is relaxed over a period indicated by arrow 1622. At time 1624, a meal is detected. The Autobolus capability delivers first and second portions of an insulin bolus in response to the meal detection. At least one safety constraint is relaxed by the AutoBolus capability for a relaxation period indicated by arrow 1626. The relaxation of at least one safety constraint resulting from the manual meal announcement is terminated as indicated by the “X” 1628. The reversion to the “standard settings” after the relaxation period indicted by arrow 1626 is shown.

FIG. 16C depicts a third plot 1630 that is like those of FIGS. 16A and 16B. However, in this example plot, at time 1632, a meal is detected. In response, the AutoBolus capability delivers first and second portions of a meal insulin bolus and relaxes at least one constraint of the “standard settings” for a period that is indicated by arrow 1634. At time 1636, the user 108 activates an activatable element to announce eating a meal. First and second portions of a meal insulin bolus are delivered to the user 108 by the drug delivery device 102. At least one safety constraint is relaxed in response for a relaxation period that is indicated by arrow 1640. The previous relaxation of at least one safety constraint resulting from the AutoBolus insulin bolus delivery is halted as indicated by the “X” 1638. The “standard settings are again employed after the relaxation period 1640 ends.

The present disclosure furthermore relates to computer programs comprising instructions (also referred to as computer programming instructions) to perform the aforementioned functionalities. The instructions may be executed by a processor. The instructions may also be performed by a plurality of processors for example in a distributed computer system. The computer programs of the present disclosure may be for example preinstalled on, or downloaded to the medicament delivery device, management device, fluid delivery device, e.g. their storage. The computer program may calculate the first portion and second portion to be delivered.

While exemplary embodiments have been described herein, various changes in form and detail may be made without departing from the intended scope of the attached claims.

Although the present invention is defined in the attached claims, it should be understood that the present invention can also (alternatively) be defined in accordance with the following embodiments:

1. A drug delivery system for delivery of drug to a user, comprising:
a storage of drug;
a needle or cannula for piercing the skin of the user to deliver the drug from the storage;
a fluid path for the drug between the storage to the needle or cannula;
a non-transitory computer-readable storage medium storing computer programming instructions; and
a processor configured to execute the computer programming instructions, wherein executing the computer programming instructions causes the processor to:
   constrain delivery of the drug to the user,
   receive glucose level values for the user,
   determine whether the user has eaten based on the received glucose level values for the user,
   provide an auto bolus capability to deliver a first portion of an drug bolus to the user responsive to the processor determining that the user has eaten, and
   relax at least one drug safety constraint for a period following the delivery of the first portion of the drug bolus so that larger doses of automated drug deliveries may be delivered subject to the relaxed at least one drug safety constraint.
      2. The drug delivery system of embodiment 1, wherein the executing of the computer programming instructions further causes the processor to deliver a second portion of the drug bolus.
      3. The drug delivery system of embodiment 2, wherein the executing of the computer programming instructions further causes the processor to determine a dose for the second portion of the drug bolus.
      4. The drug delivery system of embodiment 3, wherein the second portion of the dose is determined based at least in part in a most recent received glucose level value and drug on board for the user.
      5. The drug delivery system of embodiment 2, wherein the executing of the computer programming instructions further causes the processor to prevent delivery of another drug bolus during a cool down period following delivery of the second portion of the drug bolus.
      6. The drug delivery system of embodiment 5, wherein executing of the computer programming instructions further causes the processor to cancel the auto bolus capability if an activity mode is set wherein the activity mode being set indicates that the user is active or going to be active shortly, or if the user is in the cool down period.
      7. The drug delivery system of embodiment 1, wherein the executing of the computer programming instructions further causes the processor to cancel the relaxing of the at least one drug delivery constraint if at least one cancelation condition other than the expiration of the period is satisfied.
      8. The drug delivery system of embodiment 1, wherein the at least one cancelation condition includes at least one of differences between consecutively received blood glucose values for the user exceeding respective thresholds or an activity mode is set wherein the activity mode being set indicates that the user is active or going to be active shortly.
      9. A drug delivery system for delivery of a drug to a user, comprising:
a storage of the drug;
a needle or cannula for piercing the skin of the user to deliver the drug from the storage;
a fluid path for the drug between the storage to the needle or cannula;
a non-transitory computer-readable storage medium storing computer programming instructions; and
a processor configured to execute the computer programming instructions, wherein executing the computer programming instructions causes the processor to:
   constrain delivery of the drug to the user per current drug safety constraints;
   receive an indication of a user request to deliver a drug bolus to the user,
   responsive to the received request, deliver a first portion of the drug bolus to the user, and
   relax at least one of the current drug safety constraints for a period following the delivery of the first portion of the drug bolus so that larger doses of basal drug deliveries may be delivered if needed without being subject to the at least one of the current drug safety constraints that were relaxed.
      10. The drug delivery system of embodiment 9, further comprising an element that may be activated by the user to request delivery of the drug bolus.
      11. The drug delivery system of embodiment 9, wherein the element is one of a button, a knob, a switch, or a lever.
      12. The drug delivery system of embodiment 9, wherein the drug delivery device further comprises a user interface and the element is a user interface element.
      13. The drug delivery system of embodiment 9, wherein the executing of the computer programming instructions further causes the processor to deliver a second portion of the drug bolus.
      14. The drug delivery system of embodiment 9, wherein the executing of the computer programming instructions further causes the processor to determine a dose for the second portion of the drug bolus.
      15. The drug delivery system of embodiment 9, wherein the executing of the computer programming instructions further causes the processor to prevent delivery of another drug bolus during a cool down period following delivery of the second portion of the drug bolus.
      16. The drug delivery system of embodiment 9, wherein the executing of the computer programming instructions further causes the processor to cancel the relaxing of the at least one drug safety constraint if differences between consecutively received blood glucose values for the user exceed respective thresholds or if an activity mode is set wherein the activity mode being set indicates that the user is active or going to be active shortly.
      17. A drug delivery device, comprising:
a drug reservoir storing a drug;
a needle or cannula for piercing skin of a patient, said needle or cannula being hollow so as to serve as a conduit for delivering the drug to the user;
a non-transitory computer-readable storage storing computer programing instructions for controlling operation of the drug delivery device;
a processor for executing the computer programming instructions to cause the processor to:
   constrain delivery of the drug to the user per current drug delivery constraints;
   deliver a first portion of the drug bolus to the user, and
   relax at least one of the current drug safety constraints for a period following the delivery of the first portion of the drug bolus so that larger doses of basal drug deliveries may be delivered if needed without being subject to the at least one current drug safety constraints that were relaxed.
      18. The drug delivery device of embodiment 17, wherein multiple of the current drug safety constraints are relaxed.
      19. The drug delivery device of embodiment 17, wherein the drug safety constraints include at least one of a maximum amount of drug that can be delivered to the user from the drug delivery device in an operational cycle of the drug delivery device, a maximum amount of drug that can be delivered to the user from the drug delivery device in a specified number of operational cycles of the drug delivery device, a current setpoint for glucose level of the user, a maximum level of drug on board for the user, and a penalty amount in a cost function for extra drug delivery.
      20. The drug delivery device of embodiment 17, wherein the executing of the computer programming instructions further causes the processor to deliver a second portion of the drug bolus at a fixed time after delivery of the first portion of the drug bolus.
      21. A method performed by a processor of a drug delivery system, wherein the method comprises:
receiving glucose level values for the user, and/or other analyte level values,
determining whether the user has eaten based on the received analyte level values for the user, an auto bolus capability to determine a first portion of a drug bolus to be delivered to the user responsive to determining that the user has eaten,
determining that the drug delivery system has delivered the first portion to the user, and relaxing at least one drug safety constraint for a period following the determination that the first portion of the drug bolus has been delivered, wherein relaxing the at least one drug safety constraint allows determination of larger doses of basal drug deliveries to be delivered if needed without being subject to the at least one drug safety constraints that were relaxed.
   22. The method of embodiment 21, wherein the method further comprises determining a dose for the second portion of the drug bolus to be delivered to the user.
   23. The method of embodiment 22, wherein the second portion of the dose is determined based at least in part on a most recent received analyte level value and drug on board for the user.
   24. The method of embodiment 22 or 23, wherein the method further comprises determining that the second portion of the drug bolus has been delivered.
   25. The method of embodiment 24, wherein the method further comprises preventing delivery of another drug bolus during a cool down period following the determination that the second portion of the drug bolus has been delivered and/or sending instructions to the drug delivery device indicating that another drug bolus shall not be delivered within the cool down period following the determination that the second portion of the drug bolus has been delivered.
   26. The method of any one of embodiments 21 to 25, wherein the method further comprises canceling the auto bolus capability if an activity mode is set wherein the activity mode being set indicates that the user is active or going to be active shortly or if the user is in the cool down period.
   27. The method of any one of embodiments 21 to 26, wherein the method further causes the processor to cancel the relaxing of the at least one drug delivery constraint if at least one cancelation condition other than the expiration of the period is satisfied.
   28. The method of embodiment 26, wherein the cancelation condition(s) may include at least one of a difference between consecutively received blood glucose values for the user that exceeds a threshold or an activity mode is set wherein the activity mode being set indicates that the user is active or going to be active shortly.
   29. A method performed by a processor of a drug delivery system, wherein the method comprises:
receiving an indication of a user request to deliver a drug bolus immediately to the user,
responsive to the received request, determining a first portion of the drug bolus to be delivered the user,
determining that the first portion of the drug bolus has been delivered by the drug delivery system, and
relaxing at least one of a current drug safety constraint for a period following the delivery of the first portion of the drug bolus, wherein relaxing the at least one drug safety constraint allows determination of larger doses of basal drug deliveries to be delivered if needed without being subject to the at least one drug safety constraints that were relaxed.
   30. A method performed by a processor of a drug delivery system, wherein the method comprises determining a first portion of a drug bolus to be delivered the user,
determining that the first portion of a drug bolus has been delivered by the drug delivery system, and
relaxing at least one of the current drug safety constraints for a period following the delivery of the first portion of the drug bolus, wherein relaxing the at least one drug safety constraint allows determination of larger doses of basal drug deliveries to be delivered if needed without being subject to the at least one drug safety constraints that were relaxed.

1. A drug delivery system, comprising:
a non-transitory computer-readable storage medium storing computer programming instructions; and
a processor configured to execute the computer programming instructions, wherein executing the computer programming instructions causes the processor to:
receive glucose level values for the user,
determine whether the user has eaten based on the received glucose level values for the user, and
programmatically trigger delivery of a bolus of drug from the drug delivery system responsive to determining that the user has eaten.

2. The drug delivery system of claim 1, wherein the bolus of drug is delivered in at least two portions separated by time.

3. The drug delivery system of claim 2, wherein the bolus of drug is delivered in two portions such that a first portion is delivered at a first operational cycle of the drug delivery system and a second portion is delivered at a second operational cycle of the drug delivery system that is subsequent to the first operational cycle.

4. The drug delivery system of claim 3, wherein the executing of the computer programming instructions causes the processor to determine a dose for the first portion and a dose for the second portion.

5. The drug delivery system of claim 1, wherein the drug comprises insulin, a glucagon-like peptide-1 (GLP-1) receptor agonist, or a coformulation thereof.

6. The drug delivery system of claim 1, wherein the non-transitory computer-readable storage medium and the processor are part of a drug delivery device.

7. The drug delivery system of claim 1, wherein the executing of the computer programming instructions causes the processor to prohibit detecting that the user has again eaten for a period after the detecting that the user has eaten.

8. A method performed by a processor of a drug delivery system that includes a drug delivery device, comprising:
receiving glucose level values for a user of the drug delivery system;
determining whether the user has eaten based on the received glucose level values for the user; and
programmatically triggering delivery of a bolus of drug from the drug delivery device responsive to determining that the user has eaten.

9. The method of claim 7, wherein the bolus of drug is triggered to be delivered in at least two portions separated by time.

10. The method of claim 9, wherein the bolus of drug is triggered to be delivered in two portions such that a first portion is delivered in a first portion at a first operational cycle of the drug delivery system and a second portion at a second operational cycle of the drug delivery system that is subsequent to the first operational cycle.

11. The method of claim 10, further comprising the processor determining a dose for the first portion and a dose for the second portion.

12. The method of claim 8, wherein the drug comprises insulin, a glucagon-like peptide-1 (GLP-1) receptor agonist, or a coformulation thereof.

13. The method of claim 8, further comprising the processor prohibiting the detecting that the user has again eaten for a period after the detecting that the user has eaten.

14. A drug delivery system, comprising:
an activatable element for a user to request delivery of a drug bolus;
a processor configured for:
detecting that the activatable element has been activated,
triggering delivery of at least a portion of the drug bolus upon detecting the activation of the activatable element,
responsive to the triggering of the delivery, relaxing at least one drug safety constraint that limits how rapidly and/or how much drug is delivered.

15. The drug delivery system of claim 14, wherein the activatable element is an element that informs the system that a user has eaten a meal.

16. The drug delivery device of claim 14, wherein the drug lowers a glucose level of the user and wherein the processor is further configured to terminate the relaxing of the at least one drug safety constraints if there is a substantial decreasing glucose level trend.

17. The drug delivery system of claim 14, wherein the processor is further configured to terminate the relaxing of the at least one drug safety constraint after a specified period.

18. The drug delivery system of claim 17, wherein the processor is further configured to prevent further relaxing of the drug safety constraints responsive to activation of the activatable element for a subsequent period following the specified period.

19. The drug delivery system of claim 14, wherein the drug lowers glucose level of the user and wherein the processor is further configured to terminate the relaxing of the drug safety constraint when the processor is aware that the user is exercising.

20. The drug delivery system of claim 14, further comprising an on-body drug delivery device.
